(19) United States
(12) Patent Application Publication
(10) Pub. No.: US 2023/0111664 A1
(43) Pub. Date: Apr. 13, 2023

(54) HAND-HELD RADAR SYSTEM TO MEASURE INTRAOCULAR PRESSURE AND TO ASSESS EYE DISEASES AND METHOD THEREFOR

(71) Applicant: Natural Eye Care, Inc., New Paltz, NY (US)

(72) Inventors: Raymond Stanley Kasevich, Mt. Washington, MA (US); Mark Grossman, New Paltz, NY (US); Michael Edson, Yonkers, NY (US)

(21) Appl. No.: 17/941,159

(22) Filed: Sep. 9, 2022

Related U.S. Application Data
(60) Provisional application No. 63/360,536, filed on Oct. 8, 2021.

(51) Int. Cl. A61B 3/16

(52) U.S. Cl. CPC A61B 3/165; A61B 2562/0228; A61B 2562/0247

(57) ABSTRACT

A system and method to measure the intraocular pressure (IOP) of an eye and reflected impedance of an eye based on the generation of a source of electromagnetic wave energy with a radar generating device, creating a pattern of the generated electromagnetic wave energy at a predetermined frequency and radiating the pattern of electromagnetic wave energy onto a surface of an eye and within, from a distance, and receiving reflected energy back from the surface of the eye, converting the reflected wave energy into a Smith Chart display data format that can process the electromagnetic resonance reflection properties of the eye and display these reflection properties for IOP and for assessing other eye diseases as well as sensing indicators of brain diseases.

### CROSS-REFERENCE TO RELATED APPLICATIONS

This application claims priority from Provisional Patent Application No. 63/360,536, filed on Oct. 8, 2021, in the United States Patent and Trademark Office, the disclosure of which is incorporated herein in its entirety by reference.

### STATEMENT REGARDING FEDERALLY SPONSORED RESEARCH OR DEVELOPMENT

Not applicable.

### COPYRIGHT NOTICE

A portion of this disclosure contains material which is subject to copyright protection. The copyright owner has no objection to the photocopy reproduction by anyone of the patent document or the patent disclosure in exactly the form it appears in the Patent and Trademark Office patent file or records, but otherwise reserves all copyright rights whatsoever. 37 C. F. R 1.71(d).

### BACKGROUND OF THE INVENTIVE CONCEPT

### 1. Field of the Invention

The present inventive concept relates to a wireless hand-held system and method for measuring intraocular pressure (IOP) of an eye to determine whether eye diseases are present and to measure impedance reflection properties of other parts of the eye to detect other diseases of an eye. More particularly, the present general inventive concept relates to a wireless hand-held system and method to emit an electromagnetic wave incident on an eye and to receive back reflected energy back from the eye to determine whether eye diseases, such as glaucoma, cataracts, floaters and other existing eye diseases are present. The present general inventive concept also provides a wireless hand-held system and method for emitting an electromagnetic wave incident on an eye and to receive reflected energy back from the eye to sense a presence of beta amyloid plaque in an eye to determine whether Alzheimer's may be present, or to sense the presence any deterioration of the myelin sheath covering the nerves in the back of the eyes to determine whether multiple sclerosis (MS) may be present.

### 2. Description of the Related Art

Ocular diseases (diseases of the eyes), especially glaucoma, are quite common among people of all ages. Glaucoma is well known to be detected by measuring an increase in the intraocular pressure (IOP) of the human eye. If glaucoma is detected during early stages, it can be treated, thus saving one's vision. Glaucoma is the second leading cause of blindness in the world. 90% of glaucoma is called “Open-Angle Glaucoma” where the eye pressure (IOP) remains above normal overtime or spikes, which often results in damage to the optic nerve if not treated under a doctor's supervision as needed, particularly as if affects peripheral vision. A commonly known measurement of intraocular pressure is tonometry, which consists of a process of numbing one's eyes and then touching the surface of one's eye with a device that glows with a blue light and applies a pressure to the eye. These devices requires a professional to use, so cannot be applied by a layperson. Further, TOP varies during a 24-hour period as aqueous humor production in a human's eye changes, where most people tend to reach their peak TOP in the morning, while others reach their peak in the afternoon, evening or during sleep. The goal of daily reading of TOP is to better help the doctor manage the patient's glaucoma treatments and alert the patient to contact his/her eye doctor in the event of TOP spikes.

In applanation tonometry, the corneal curvature of one's eye is flattened or applaned by a flat piston. The piston is generally of a certain known weight and the area of the applanation is determined by an indirect method or it may be of a known surface area and the intraocular pressure necessary to applane that area is determined by calculating the force necessary to applane that area. Apparatuses that use pistons of a known surface area include: a) Machay-Marg which uses electronic means for a readout; b) Goldman slit lamp which uses an optical means for readout; and c) the Tonour which uses a pumped pressure readout. All of these known methods require both a topical anesthetic and a device which contacts the eye, thereby risking of corneal abrasion. Further each of these methods are complicated, expensive and difficult to use. These units are large and cumbersome, and require a practitioner to use on the patient.

Another known method of testing for TOP is called “the air puff method,” using an air puff tonometer. The air puff tonometer also uses an applanation method where a standardized puff of air flattens a portion of the cornea of one's eye, while there is no actual contact with the eye. In this method a central air planum is used, which requires a light emitter at one side thereof and a light detector on an opposite side thereof. Corneal applanation is measured by collecting light reflected from the central cornea. This system records the force of air required to flatten the cornea and displays the TOP that corresponds to that force. The air puff tonometer must be used at a set distance from the cornea, and the instrument incorporates an optical alignment system. A well-known problem with the air puff method is the overestimation of low intraocular pressures and the underestimation of high intraocular pressures. The underestimation would result in false negative results.

U.S. Pat. No. 9,795,295 by Cohen discloses a tonometer for checking the intraocular pressure through an eyelid, as illustrated in FIG. 1. Here a mechanical TOP monitor includes a cylinder 2 fitted with an ocular plate 11 attached to an inner tube 8, which moves against the pressure of a spring 15. The cylinder interior comprises a mechanism for transducing pressure, detecting intraocular pressure and a signaling component for indicating when a set intraocular pressure is exceeded. Here the ocular plate 11 must be rested on the outer surface of one's eyelid or eye, and then a force, provided by the user, compresses the internal spring 15. The force passes through the eyelid to the surface of the eye, where a visual indicator will indicate a pass or fail of the intraocular pressure reading. Although this device is suitable for use without any on-site supervision by a skilled person and does not require any prior application of anesthesia, the device by Cohen requires contact with one's eye or eyelid, and it is also required to apply a mechanical force on the eye.

Accordingly, there is a need for a mobile hand-held tonometer and method therefor that can measure intraocular pressure (IOP) of an eye without making contact with the eye and without applying direct pressure against the eye.

There is also a need for a mobile hand-held device and method therefor that can assess for eye diseases including glaucoma, cataracts, floaters, and other existing eye diseases, without making contact with the eye and without applying direct pressure against the eye.

There is also a need for a mobile hand-held device and method therefor that can vary a frequency of a transmitted electromagnetic wave in order to detect different types of diseases arising in the eyes.

There is also a need for an inexpensive mobile hand-held device and method therefor for measuring intraocular pressure based on the ability of an eye to re-radiate in the presence of an electromagnetic wave.

There is also a need for an inexpensive mobile hand-held device and a method therefor that can enable a person to perform daily readings of TOP of their eyes to better help a doctor manage the user's glaucoma treatments and alert the user to contact his/her eye doctor in the event of TOP spikes throughout each day.

### SUMMARY OF THE INVENTIVE CONCEPT

The present general inventive concept provides a wireless system and method for measuring intraocular pressure (IOP) of an eye to determine whether eye diseases are present. More particularly, the present general inventive concept provides a wireless system and method to emit an electromagnetic wave incident on an eye to determine whether eye diseases, such as glaucoma, cataracts, floaters, MS vision problems, and other existing eye diseases are present.

The present general inventive concept also provides a wireless system and method for measuring intraocular pressure (IOP) of an eye to determine whether Alzheimer's is present.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a mobile hand-held radar tonometer system to measure the intraocular pressure (IOP) of an eye, comprising: a microwave source configured to generate a source of electromagnetic wave energy, to measure reflected wave energy from a surface of an eye and to convert the measured reflected wave energy into a Smith Chart display data format; a radar antenna configured to create a pattern of the electromagnetic wave energy from the microwave source at a predetermined frequency to be radiated onto an eye and to simultaneously receive reflected energy back from the eye; a coaxial cable connected between the microwave source and the radar antenna to communicate electromagnetic wave energy therebetween; and an on-board computer processor to store and transmit the Smith Chart display formatted data to an external display device configured to display the Smith Chart impedance of the eye.

In an exemplary embodiment, the predetermined frequency of the electromagnetic wave can be the frequency of resonance of the eye.

In another exemplary embodiment, the radar antenna is a microwave antenna dipole configured to act as a load electrical connection to the microwave source to provide an electromagnetic reflection signal from the eye for intraocular data processing.

In another exemplary embodiment, the frequency of the created pattern of the electromagnetic wave energy is adjustable to direct the electromagnetic energy distributions into different components of the eye.

In still another exemplary embodiment, the system can further comprise a mobile display device including software configured to display the Smith Chart impedance of the eye using the transmitted Smith Chart display formatted data.

In still another exemplary embodiment, the radar antenna is configured to be positioned at approximately 1 mm away from the surface of the eye.

In yet another exemplary embodiment, the microwave source is a nano vector network analyzer (VNA).

The foregoing and/or other features and utilities of the present general inventive concept may also be achieved by providing a method of measuring the intraocular pressure (IOP) of an eye, the method comprising: generating a source of electromagnetic wave energy with a radar generating device; creating a pattern of the generated electromagnetic wave energy at a predetermined frequency and radiating the pattern of electromagnetic wave energy onto a surface of an eye while simultaneously receiving reflected energy back from the surface of the eye; and converting the reflected wave energy into a Smith Chart display data format that can display impedance reflection properties of the eye.

In an exemplary embodiment, the predetermined frequency of the electromagnetic wave is the frequency of resonance of the eye.

In another exemplary embodiment, the predetermined frequency of the created pattern of the electromagnetic wave energy is adjusted to direct the electromagnetic energy distributions into different components of the eye.

In still another exemplary embodiment, the pattern of the generated electromagnetic wave energy is radiated onto the surface of the eye from a distance of approximately 1 mm away from the eye.

The foregoing and/or other features and utilities of the present general inventive concept may also be achieved by providing a method of measuring reflective impedance of an eye, the method comprising: generating a source of electromagnetic wave energy; creating a pattern of the electromagnetic wave energy at a predetermined frequency and radiating the pattern of the electromagnetic wave energy onto a surface of an eye and within the eye from a distance; receiving reflected energy back from the surface of the eye; converting the reflected wave energy into a Smith Chart display data format that can process the electromagnetic resonance reflection properties of the eye; and displaying the resonance reflection properties for assessment of TOP and other eye diseases.

In an exemplary embodiment, the predetermined frequency of the electromagnetic wave is set to the frequency of resonance of the eye to assess for TOP.

In another exemplary embodiment, the predetermined frequency of the created pattern of the electromagnetic wave energy is adjusted to direct the electromagnetic energy distributions into different components of the eye depending on the type of eye disease to be assessed.

In still another exemplary embodiment, the pattern of the generated electromagnetic wave energy is radiated onto the surface of the eye from a distance of approximately 1 mm away from the eye.

### BRIEF DESCRIPTION OF THE DRAWINGS

These and/or other features and utilities of the present inventive concept will become apparent and more readily appreciated from the following description of the embodiments, taken in conjunction with the accompanying drawings of which:

FIG. 1 illustrates a conventional hand-held tonometer for checking the intraocular pressure of an eye;

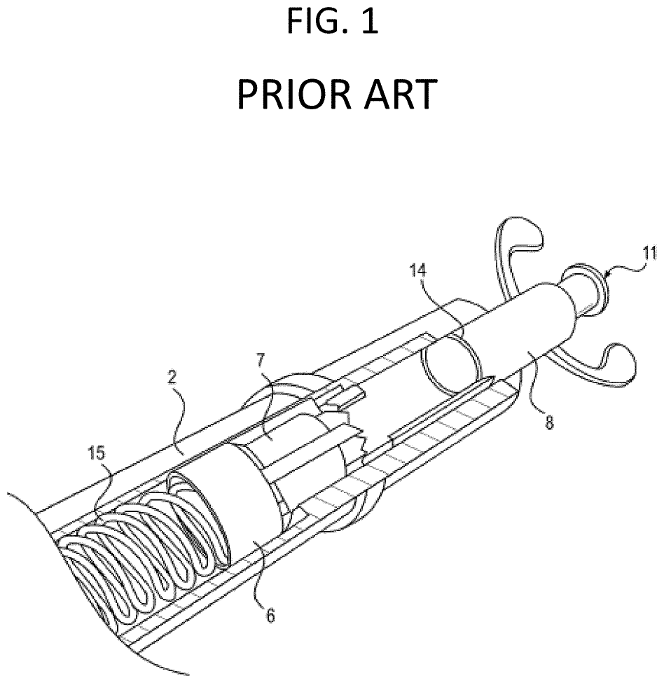

FIG. 2 illustrates a hand-held wireless system for measuring intraocular pressure (IOP) of an eye, according to an exemplary embodiment of the present inventive concept;

FIG. 2A illustrates a radar antenna dipole of the system of FIG. 1, according to an exemplary embodiment of the present inventive concept;

FIG. 3 illustrates a method of measuring intraocular pressure (IOP) in accordance with an exemplary embodiment of the present inventive concept;

FIG. 4 illustrates an example of reading the results of the method of measuring intraocular pressure in accordance with the exemplary embodiment of FIG. 2;

FIG. 5 illustrates different patterns of soliton energy in an eye created by transmitting electromagnetic waves incident on the eye using the hand-held wireless system of FIG. 1 at different frequencies.

FIG. 6 illustrates an example of a solitonic wave being emitted at a higher frequency than resonant frequency provided by the radar antenna dipole of the system of FIG. 1 to assess for other diseases of the eye, as well as for Alzheimer's disease.

The drawings illustrate a few exemplary embodiments of the present inventive concept, and are not to be considered limiting in its scope, as the overall inventive concept may admit to other equally effective embodiments. The elements and features shown in the drawings are to scale and attempt to clearly illustrate the principles of exemplary embodiments of the present inventive concept. In the drawings, reference numerals designate like or corresponding, but not necessarily identical, elements throughout the several views.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below in order to explain the present general inventive concept while referring to the figures. Also, while describing the present general inventive concept, detailed descriptions about related well-known functions or configurations that may diminish the clarity of the points of the present general inventive concept are omitted.

It will be understood that although the terms “first” and “second” are used herein to describe various elements, these elements should not be limited by these terms. These terms are only used to distinguish one element from another element. Thus, a first element could be termed a second element, and similarly, a second element may be termed a first element without departing from the teachings of this disclosure.

Expressions such as “at least one of,” when preceding a list of elements, modify the entire list of elements and do not modify the individual elements of the list.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the detailed description of the invention. Thus, the terms used herein have to be defined based on the meaning of the terms together with the description throughout the specification.

Also, when a part “includes” or “comprises” an element, unless there is a particular description contrary thereto, the part can further include other elements, not excluding the other elements. In the following description, terms such as “unit” and “module” indicate a unit to process at least one function or operation, wherein the unit and the block may be embodied as hardware or software or embodied by combining hardware and software.

Hereinafter, one or more exemplary embodiments of the present general inventive concept will be described in detail with reference to accompanying drawings.

Exemplary embodiments of the present general inventive concept are directed to a wireless system and method for measuring intraocular pressure (IOP) of an eye to determine whether eye diseases, such as glaucoma, cataracts, floaters, MS vision problems, as well as other existing eye diseases are present. The present general inventive concept is also directed to a wireless system and method for measuring intraocular pressure (IOP) of an eye to determine whether Alzheimer's is present.

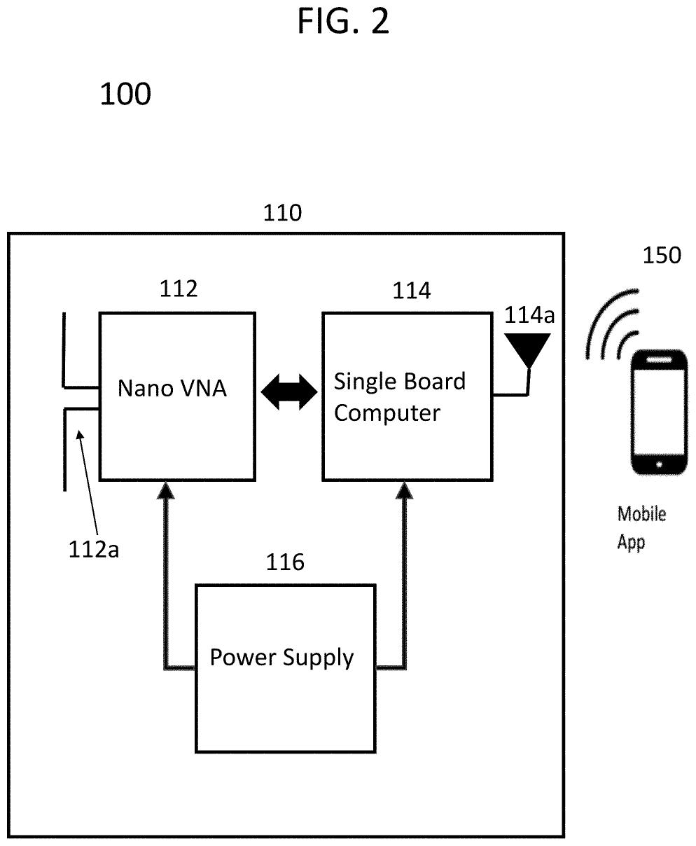

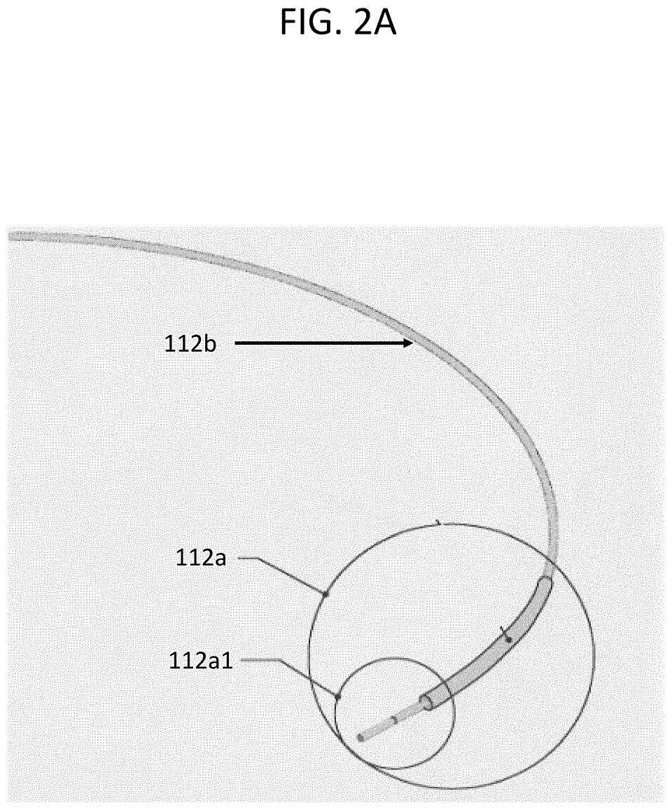

FIG. 2 illustrates a mobile hand-held radar tonometer system 100 to measure the intraocular pressure (IOP) of an eye, according to an exemplary embodiment of the present inventive concept. The mobile hand-held radar system 100 according to the present exemplary embodiment can include a nano Vector Network Analyzer (VNA) 112, which is configured to generate a source of electromagnetic wave energy, to measure reflected wave energy from a surface of an eye and to convert the measured reflected wave energy into VNA Smith Chart display data. Alternative equivalent devices which can supply energy for assessment and detection of eye diseases can be used, such as for example a microwave source. The mobile hand-held radar system 100 illustrated in FIG. 2 can also include a single board computer processor 114 for data recording and analysis, to convert the measured reflective wave energy into the Smith Chart data format and to provide Wi-Fi and Bluetooth® communication capabilities with a mobile phone or other personal display device in order to transmit VNA Smith Chart display data. Both the VNA 112 and the computer 114 can be powered by a small battery power supply 116 or other efficient power supply.

Referring to FIG. 2 and FIG. 2A, a radar antenna 112a, such as a miniature dipole radar antenna, can be connected to the VNA 112 via a flexible microwave coaxial cable 112b. The radar antenna 112a can include an antenna dipole 112a1 having a length of approximately 2.5 cm. The radar antenna 112a is disposed at a first end of the flexible coaxial cable 112b opposite a second end of the flexible coaxial cable that is connected to the VNA 112. The radar antenna 112a creates a pattern of electromagnetic energy that can focus on the surface of an eye. The radar antenna 112a transmits the electromagnetic wave energy at a set frequency. The set frequency of the transmitted electromagnetic wave energy will depend on what type of disease is desired to be detected within the eye, and can be controlled via the VNA 112, as will be discussed in more detail below. Other frequencies of the transmitted electromagnetic wave energy can be set to sense such things as beta amyloid plaque that occurs in specific parts of the eye, where beta amyloid plaque can be an indicator of Alzheimer's disease, or to sense such things as the presence of a myelin sheath covering the nerves in the ack of the eyes, which is an indicator of the presence of multiple sclerosis (MS). In other words, the frequency of the electromagnetic wave can be adjusted through the VNA 112 based on what is intended to be detected from receiving back reflected impedance energy from the eye. It is to be noted that alternative equivalent types of antennas for sensing the electromagnetic properties of the eye can be used, such as electric and magnetic types of sensing antennas, which provide the intended purposes as described herein, without departing from the principles, spirit and scope of the present inventive concept.

The radar antenna 112a can radiate the generated electromagnetic wave energy to an eye through a dipole 112a1. The radar antenna dipole 112a1 is preferably held in a position over the eye and at a distance from the eye of approximately 1 mm. It is to be noted that alternative equivalent types of antennas that can perform the intended purposes of radiating electromagnetic waves and receiving back reflected energy, as described herein, can be used without parting from the principles, spirit and scope of the general inventive concept.

The radar antenna 112a can be disposed on a frame of eyeglasses such that upon wearing the glasses the radar antenna 112a is naturally positioned approximately 1 mm from the eye of a person wearing the eyeglasses. It is noted that the radar antenna 112a can alternatively be disposed on other devices which will aid in positioning the radar antenna 112a approximately 1 mm from the eye.

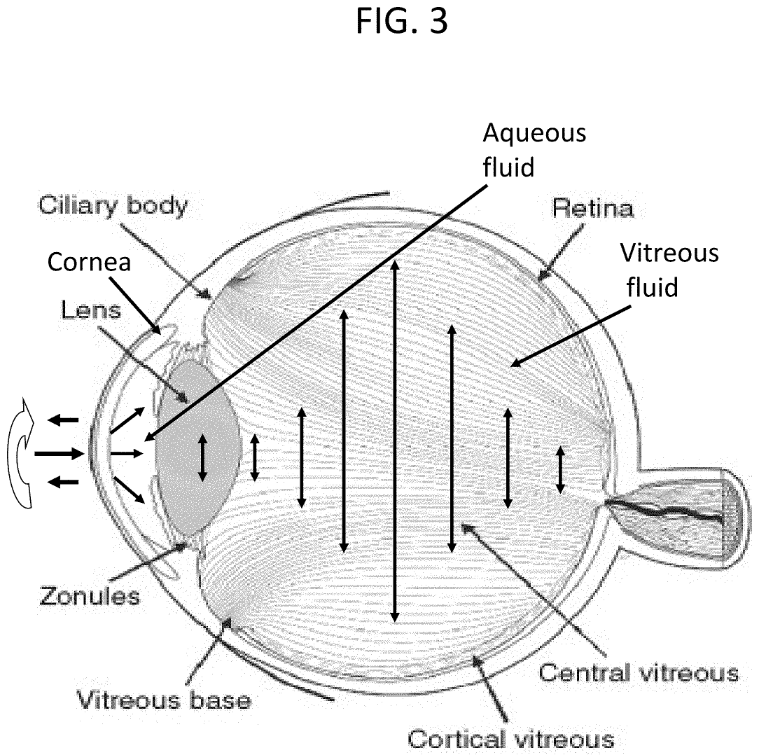

Once the radar antenna 112a is positioned adjacent to the eye the radiated electromagnetic wave becomes incident to the eye. The radar antenna 112a is preferable placed approximately 1 mm away from the eye and should not contact the eye. In an exemplary embodiment, the radar antenna 112a radiates the electromagnetic wave at a frequency of resonance of the eye (approximately 855-860 megahertz) which is usually best for measuring intraocular pressure (IOP) for normal adult size eyes. In other words, if the axial eye length (AEL), measured from front to back, is approximately ½ the wavelength of the radiated electromagnetic wave, a resonance will occur such that as the electromagnetic wave is incident to the eye most of the absorbed electromagnetic energy of the electromagnetic wave will exist at and near the center of the eye volume and within the vitreous humor fluid of the eye (see FIG. 3). It has been determined that the mean range of eye axial lengths is 21.8 mm to 24 mm for people in the range of age from 21 to 91 years old.

The radar antenna 112a will receive back from the eye a reflected energy wave at a fixed frequency. This reflection occurs because the eye surface has developed a dielectric impedance from the radar energy absorbed by the eye. As the frequency of the electromagnetic wave is transmitted at the frequency of resonance of the eye (½ wavelength of the axial eye length (AEL), the impedance will become purely resistive in nature. The eye surface impedance determines the amplitude and phase of the reflected wave as a percentage of the incident wave generated at the radar antenna 112a. The nearly spherical eye volume acts not only as an absorber of the energy of the incident electromagnetic wave but also as a reflector of this incident electromagnetic wave energy. As a result, the radar antenna 112a sees the eye by the fact that it directly receives back a reflected wave signal. In effect, the eye will act as an electrically resistive load on the radar antenna 112a at the first resonant frequency of the eye noted on a Smith Chart. When transmitting the electromagnetic wave energy at different selected frequencies incident on an eye, a reflective energy received back will provide a broad range of reflection impedance of the eye, which can be displayed on a Smith Chart for assessment of the eye.

FIG. 3 illustrates an example of the radar antenna 112a transmitting an electromagnetic wave into an eye, and the electric field directions and relative amplitudes of the electromagnetic wave (illustrated by the arrows) both outside and inside the eye. As illustrated in FIG. 3, the absorbed electromagnetic wave energy undergoes internal reflections in the eye volume and thus generates a volumetric pattern of electric and magnetic field energy. The ½ wavelength of the internal reflections is selected to create resonance in the eye volume. It is noted that the incident wave from the radar antenna has a much larger wavelength as compared to the wavelength in the eye. Essentially the eye fluid compresses the wavelength in the eye because of the fluid dielectric properties. Thus, when the frequency of the electromagnetic wave is adjusted such that ½ of the wavelength of the electromagnetic wave is substantially the same size as the axial eye length (AEL), an eye resonance will occur, as pointed out above, such that most of the absorbed electromagnetic energy will exist at and near the center of the eye volume (mostly within the vitreous humor fluid), while a small amount of the absorbed energy occurs at the eye boundary (cornea), giving the eye the ability to reflect waves back to the radar antenna by a cornea surface impedance established by the wave internal reflections. This impedance is purely resistive in nature at the resonance frequency. The surface impedance is a measure of the electromagnetic reflectivity of the eye. The radar antenna 112a basically detects the eye as a small resistance target at the first observed IOP resonance data point on the Smith Chart data display.

The intraocular pressure (IOP) is defined as the fluid pressure of the eye given by (F/C+P), where F represents aqueous flow rate, C represents aqueous outflow, and P is the episcleral venous pressure). It is desirable to choose the radar antenna 112a operating frequency for IOP such that ½ wavelength of the electromagnetic wave inside the eye is approximately equal to the axial eye length (AEL) since AEL correlates with IOP. Resonance provides the most sensitivity to IOP measurement using the mobile hand-held radar tonometer system 100 illustrated in FIG. 2. The choice of operating frequency of the electromagnetic wave having ½ wavelength equal to the AEL ensures measurement accuracy and sensitivity to small changes in IOP.

Still referring to FIG. 3, both an incident radar signal (arrow pointing left to the cornea) and a signal reflected back to the radar antenna at resonance (arrows pointing right away from the cornea) are illustrated. The first resonant frequency condition maximizes the wave power in the eye with the least amount reflected back to the radar antenna. The distribution of wave power from the front to the back of the eye at resonance will be maximum at the center of the eye and minimum at eye volume boundaries. During testing with the mobile hand-held radar tonometer system 100 this distribution has been experimentally shown to provide a maximum of sensitivity to IOP measurement. The reflected wave received by the antenna 112a is in the form of signal voltage conveyed to the miniature vector network analyzer (VNA) 112 by the miniature coaxial transmission line 112b connecting the radar antenna terminals of the radar antenna 112a to the vector network analyzer (VNA) 112.

The resonant frequency corresponds to the electrical ½ wavelength of the electromagnetic wave oscillation inside the eye primarily based on the high dielectric vitreous humor (VH) volume. The electromagnetic absorption is extensive in the vitreous humor volume (VH) because of its high dielectric constant compared to other parts of the eye, such as the retina, cornea, etc. The relative dielectric constant of the relatively large VH volume is approximately 69 with negligible electrical conductivity. The eye essentially scatters and absorbs the incident radar energy. Some eye structural components may interact with the electromagnetic signal absorbed by the eye depending on their dielectric properties, geometry and the positioning of the radar antenna 112a next to the eye surface. In the practice of ophthalmology, the employment of a well-known exponential (Friedenwald) relationship between changes in volume related to changes in eye internal pressure was employed for the IOP data processing using measured radar data. However, this has been accomplished only by large and expensive industrial size stationary equipment. However, using the mobile hand-held radar system 100 illustrated in FIG. 2 and process described above, indications of electric fields (directions and relative amplitudes) inside and outside the eye generated by the radar antenna 112a can be obtained, as illustrated by the arrows in FIG. 3. It is to be noted that the arrows illustrated in FIG. 3 are not provided to scale but only to illustrate the electric fields both inside and outside the eye as a result of the electromagnetic wave generated and transmitted by the radar antenna 112a.

The backscattered or reflected electric field received by the radar antenna 112a and measured by the vector network analyzer (VNA) 112 provides the eye IOP data in a Smith Chart data format. This IOP data in a Smith Chart format can be transmitted from the single board computer 114 via either Wi-Fi or Bluetooth® 114a to a mobile device 150 including an App to display Smith Chart data, as illustrated in FIG. 2.

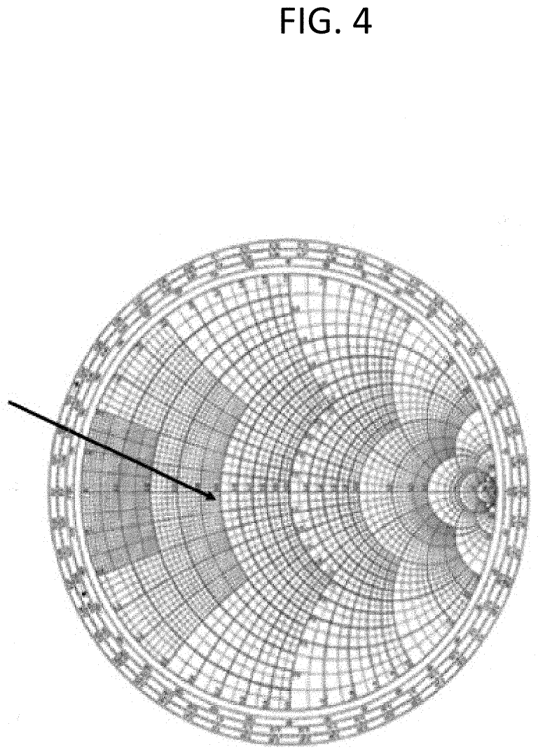

FIG. 4 illustrates the Smith Chart (SC) display of measurements which were received and converted to Smith Chart data by the nano VNA 112 after the radar antenna 112a transmitted an electromagnetic wave to be incident on an eye. Here the reflection coefficient and resistance at the resonant frequency are displayed. In the example illustrated in FIG. 4, for the typical IOP resonance measurements performed, eye resistance values of approximately less than 10 ohms were measured and are indicated along the Smith Chart horizontal axis at the tip of the arrow.

The frequency of the radar antenna 112a operation can be changed to provide a variety of three-dimensional distributions of electromagnetic field patterns inside the eye. In other words, the mobile hand-held radar system 100 can direct its electromagnetic energy distributions into different components of the eye volume based on the frequency employed relative to the eye size and orientation of the antenna 112a. In addition, the frequency employed and radar antenna radiation can be altered through antenna design, as described below. These different distributions of electric field patterns in the eye can be theoretically predicted using Mie electromagnetic scattering theory. Although the first resonance is used for successful IOP application, as pointed out above, radar tonometry applications to assess different diseases, such as for example, Alzheimer's (AZ) and multiple sclerosis (MS), can also be performed using the mobile hand-held radar system 100 illustrated in FIG. 2. More specifically, a build-up of beta amyloid plaque has been shown to be an indicator of the presence of Alzheimer's disease. Thus, by transmitting electromagnetic field energy focused on the retina (solitonic energy distribution) through frequency selection, a Smith Chart impedance value of the eye can be obtained which correlates with the build-up of beta amyloid plaque, which is an indicator of the presence of Alzheimer's. Also, the presence of a myelin sheath that covers that nerves in the back of the eye is an indicator of a risk of having multiple sclerosis (MS).

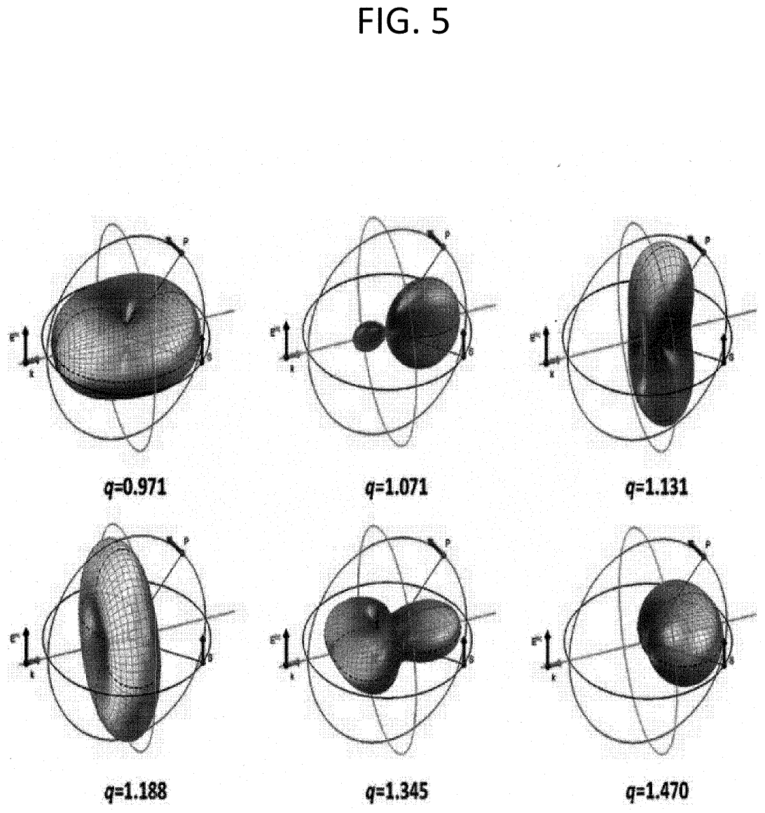

FIG. 5 illustrates six different patterns of soliton energy distributions in a dielectric sphere (i.e., the eye) with an electromagnetic wave incident on the sphere at different frequencies. These patterns show the possible internal eye distributions of electromagnetic energy using the mobile hand-held radar system 100 and process described above, at different measurement frequencies.

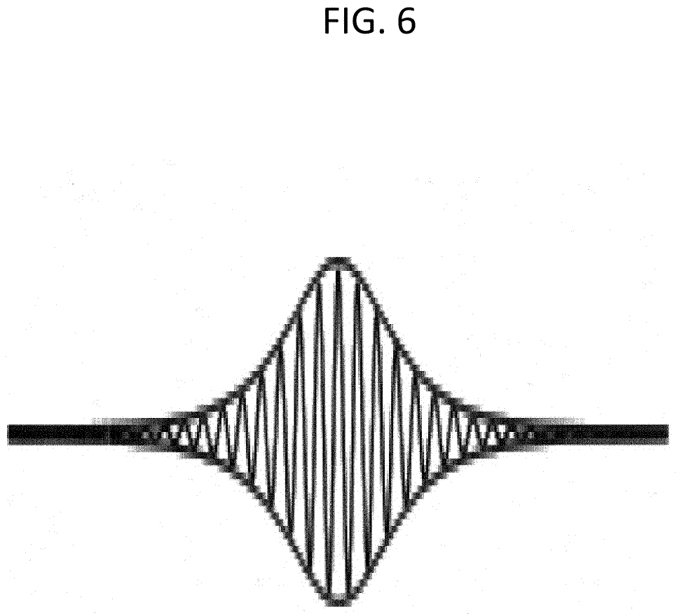

FIG. 6 illustrates a solitonic eye pattern at a frequency higher than that for TOP. In this case the radar antenna 112a that would be most effective is electrically smaller than the free space wavelength which generates and transmits the resonant frequency, such as a Hertzian dipole antenna.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

What is claimed is:

1. A mobile hand-held radar tonometer system to measure the intraocular pressure (IOP) of an eye, comprising:
a microwave source configured to generate a source of electromagnetic wave energy, to measure reflected wave energy from a surface of an eye and to convert the measured reflected wave energy into a Smith Chart display data format;
a radar antenna configured to create a pattern of the electromagnetic wave energy from the microwave source at a predetermined frequency to be radiated onto an eye and to simultaneously receive reflected energy back from the eye;
a coaxial cable connected between the microwave source and the radar antenna to communicate electromagnetic wave energy therebetween; and
an on-board computer processor to store and transmit the Smith Chart display formatted data to an external display device configured to display the Smith Chart impedance of the eye.

2. The system according to claim 1, wherein the predetermined frequency of the electromagnetic wave is the frequency of resonance of the eye.

3. The system according to claim 2, wherein the radar antenna is a microwave antenna dipole configured to act as a load electrical connection to the microwave source to provide an electromagnetic reflection signal from the eye for intraocular data processing.

4. The system according to claim 1, wherein the frequency of the created pattern of the electromagnetic wave energy is adjustable to direct the electromagnetic energy distributions into different components of the eye.

5. The system according to claim 1, further comprising:
a mobile display device comprising software configured to display the Smith Chart impedance of the eye using the transmitted Smith Chart display formatted data.

6. The system according to claim 1, wherein the radar antenna is configured to be positioned at approximately 1 mm away from the surface of the eye.

7. The system according to claim 1, wherein the microwave source is a nano vector network analyzer (VNA).

8. A method of measuring the intraocular pressure (IOP) of an eye, the method comprising:
generating a source of electromagnetic wave energy with a radar generating device;
creating a pattern of the generated electromagnetic wave energy at a predetermined frequency and radiating the pattern of electromagnetic wave energy onto a surface of an eye while simultaneously receiving reflected energy back from the surface of the eye; and
converting the reflected wave energy into a Smith Chart display data format that can display impedance reflection properties of the eye.

9. The method according to claim 8, wherein the predetermined frequency of the electromagnetic wave is the frequency of resonance of the eye.

10. The method according to claim 8, wherein the predetermined frequency of the created pattern of the electromagnetic wave energy is adjusted to direct the electromagnetic energy distributions into different components of the eye.

11. The method according to claim 8, wherein the pattern of the generated electromagnetic wave energy is radiated onto the surface of the eye from a distance of approximately 1 mm away from the eye.

12. A method of measuring reflective impedance of an eye, the method comprising:
generating a source of electromagnetic wave energy;
creating a pattern of the electromagnetic wave energy at a predetermined frequency and radiating the pattern of the electromagnetic wave energy onto a surface of an eye and within the eye from a distance;
receiving reflected energy back from the surface of the eye;
converting the reflected wave energy into a Smith Chart display data format that can process the electromagnetic resonance reflection properties of the eye; and
displaying the resonance reflection properties for assessment of TOP and other eye diseases.

13. The method according to claim 12, wherein the predetermined frequency of the electromagnetic wave is set to the frequency of resonance of the eye to assess for TOP.

14. The method according to claim 12, wherein the predetermined frequency of the created pattern of the electromagnetic wave energy is adjusted to direct the electromagnetic energy distributions into different components of the eye depending on the type of eye disease to be assessed.

15. The method according to claim 12, wherein the pattern of the generated electromagnetic wave energy is radiated onto the surface of the eye from a distance of approximately 1 mm away from the eye.
